(19) United States
(12) Patent Application Publication
(10) Pub. No.: US 2025/0001139 A1
(43) Pub. Date: Jan. 2, 2025

(54) EXPANDABLE INTRODUCER

(71) Applicant: Medtronic, Inc., Minneapolis, MN (US)

(72) Inventors: Timothy Desmond FARRELL, Tralee (IE); Chris MORAN, Mayo (IE); Marc A. ANDERSON, Barna (IE); Patricia MCAFEE, Galway (IE); Luke A. CLARKE, Galway (IE); Antonia TROTTA, Athenry (IE); Andrea Quero MARTIN, Galway (IE); Tomas Patrick KITT, San Francisco, CA (US); Ross L. WILCOX, Santa Rosa, CA (US); Patrick GRIFFIN, Galway (IE)

(73) Assignee: Medtronic, Inc., Minneapolis, MN (US)

(21) Appl. No.: 18/615,140

(22) Filed: Mar. 25, 2024

Related U.S. Application Data
(60) Provisional application No. 63/510,670, filed on Jun. 28, 2023.

(51) Int. Cl. A61M 25/06; A61M 25/00

(52) U.S. Cl. CPC A61M 25/0662; A61M 25/0023; A61M 25/0052; A61M 2025/0024; A61M 2025/0681

(57) ABSTRACT

An expandable introducer includes a hub having a distal end and a proximal end, and a sheath coupled to the hub and extending distally therefrom, the sheath defining a central lumen. The sheath is configured to radially expand from a radially unexpanded, folded state having a first diameter and to a radially expanded, unfolded state having a second diameter larger than the first diameter in response to a device passing through the central lumen. A wall of the sheath includes a thick wall region having a first wall thickness and a thin wall region having a second wall thickness smaller than the first wall thickness, wherein the thick wall region includes a coil embedded within the wall of the sheath.

### CROSS-REFERENCE TO RELATED APPLICATIONS

This application claims the benefit of U.S. Provisional Patent Application Ser. No. 63/510,670, filed Jun. 28, 2023, the entire content of which is incorporated herein by reference.

### FIELD OF THE INVENTION

The present invention relates to an expandable introducer device for providing percutaneous access to a patient's vasculature for a transcatheter device delivery system.

### BACKGROUND

An introducer device is used to provide percutaneous access to the vascular system of a patient and functions to permit the introduction and positioning of various minimally invasive delivery devices within the patient's vasculature. In general, making the smallest incision is the most desirable and leads to less complications, less trauma, and improved patient outcomes. Some delivery devices, however, are large and require large sheaths to accommodate and help deliver them to the desired site within the body.

### BRIEF SUMMARY OF THE INVENTION

In accordance with an example hereof, an expandable introducer includes a hub having a distal end and a proximal end, and a sheath coupled to the hub and extending distally therefrom, the sheath defining a central lumen. The sheath is configured to radially expand from a radially unexpanded, folded state having a first diameter and to a radially expanded, unfolded state having a second diameter larger than the first diameter in response to a device passing through the central lumen. A wall of the sheath includes a thick wall region having a first wall thickness and a thin wall region having a second wall thickness smaller than the first wall thickness, wherein the thick wall region includes a coil embedded within the wall of the sheath.

In another example hereof, in the expandable introducer of any of the previous or subsequent examples herein, the first wall thickness is about 0.4-0.7 mm.

In another example hereof, in the expandable introducer of any of the previous or subsequent examples herein, the second wall thickness is about 0.05-0.2 mm.

In another example hereof, in the expandable introducer of any of the previous or subsequent examples herein, the coil is a C-shaped coil including a plurality of circumferential portions coupled by bends.

In another example hereof, in the expandable introducer of any of the previous or subsequent examples herein, adjacent bends of the coil face opposite circumferential directions such that there is a circumferential gap between the bends facing opposite directions in the radially unexpanded, folded state.

In another example hereof, in the expandable introducer of any of the previous or subsequent examples herein, the coil is a shape memory material and is shape set to the radially unexpanded, folded state.

In another example hereof, in the expandable introducer of any of the previous or subsequent examples herein, the sheath comprises a proximal portion disposed within a lumen of the hub, an expanded portion distal of the proximal portion, a tapered portion distal of the expanded portion, an expandable coiled portion distal of the tapered portion, and an expandable non-coiled portion distal of the expandable coiled portion.

In another example hereof, in the expandable introducer of any of the previous or subsequent examples herein, the expanded portion is not foldable and is the second diameter.

In another example hereof, in the expandable introducer of any of the previous or subsequent examples herein, the tapered portion tapers in the distal direction such that in the radially unexpanded, folded state of the sheath, a proximal end of the tapered portion has the second diameter and a distal end of the tapered portion has the first diameter.

In another example hereof, in the expandable introducer of any of the previous or subsequent examples herein, wherein in the radially expanded, unfolded state, both the proximal end and the distal end of the tapered portion have the second diameter.

In another example hereof, in the expandable introducer of any of the previous or subsequent examples herein, the expandable coiled portion has the first diameter in the radially unexpanded, folded state and the second diameter in the radially expanded unfolded state.

In another example hereof, in the expandable introducer of any of the previous or subsequent examples herein, the expandable non-coiled portion has the first diameter in the radially unexpanded, folded state and the second diameter in the radially expanded, unfolded state.

In another example hereof, in the expandable introducer of any of the previous or subsequent examples herein, the coil is embedded in the wall of the sheath along the expanded portion, the tapered portion, and the expandable coiled portion.

In another example hereof, in the expandable introducer of any of the previous or subsequent examples herein, the coil is not embedded in the wall of the sheath along the proximal portion and the expandable non-coiled portion.

In another example hereof, in the expandable introducer of any of the previous or subsequent examples herein, the coil is a shape memory material, and wherein an Active A(f) temperature of the coil is higher in a proximal region of the coil than in a distal region of the coil.

In another example hereof, in the expandable introducer of any of the previous or subsequent examples herein, the coil includes a proximal expanded region embedded in the expanded portion of the sheath, a tapered region embedded in the tapered portion of the sheath, and a distal expandable region embedded in the expandable coiled portion of the sheath.

In another example hereof, in the expandable introducer of any of the previous or subsequent examples herein, the proximal expanded region and the tapered region of the coil have a first Active A(f) temperature and the distal expandable region of the coil has a second Active A(f) temperature lower than the first Active A(f) temperature.

In another example hereof, in the expandable introducer of any of the previous or subsequent examples herein, the proximal expanded region and the tapered region of the coil have a first Active A(f) temperature, a proximal portion of the distal expandable region of the coil has a second Active A(f) temperature lower than the first Active A(f) temperature, and a distal portion of the distal expandable region of the coil has a third Active A (f) temperature lower than the second Active A(f) temperature.

In another example hereof, in the expandable introducer of any of the previous or subsequent examples herein, the coil comprises a wire, wherein at least one end of the wire is formed into a pigtail.

In another example hereof, in the expandable introducer of any of the previous or subsequent examples herein, the coil comprises a wire, wherein at least one end of the wire is disposed closer to a central longitudinal axis of the sheath than a middle portion of the wire.

In another example hereof, in the expandable introducer of any of the previous or subsequent examples herein, the coil comprises a wire, wherein at least one end of the wire is laser cut such that the at least one end has a smooth profile.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other features and advantages of the present disclosure will be apparent from the following description of embodiments hereof as illustrated in the accompanying drawings. The accompanying drawings, which are incorporated herein and form a part of the specification, further serve to explain the principles of the present disclosure and to enable a person skilled in the pertinent art to make and use the embodiments of the present disclosure. The drawings may not be to scale.

FIG. 1A shows a schematic side view of an expandable introducer in an unexpanded, folded state according to embodiments hereof.

FIG. 1B shows a schematic side view of the expandable introducer of FIG. 1A in an expanded, unfolded state according to embodiments hereof.

FIG. 2A shows a schematic cross-section of a sheath of the expandable introducer of FIG. 1A taken along line A-A of FIG. 1A according to embodiments hereof.

FIG. 2B shows a schematic cross-section of a sheath of the expandable introducer of FIG. 1A taken along line B-B of FIG. 1B according to embodiments hereof.

FIG. 3A shows a schematic cross-section of a sheath of the expandable introducer of FIG. 1A taken along line A-A of FIG. 1A according to embodiments hereof.

FIG. 3B shows a schematic cross-section of a sheath of the expandable introducer of FIG. 1A taken along line B-B of FIG. 1B according to embodiments hereof.

FIG. 4A shows a schematic side view of a coil of the sheath of the introducer of FIG. 1A in an unexpanded, folded state according to embodiments hereof.

FIG. 4B shows a schematic perspective view of a portion of the coil of FIG. 4A.

FIG. 5 shows a schematic close-up view of a portion of the coil of FIG. 4A in an unexpanded state according to embodiments hereof.

FIG. 6 shows a schematic close-up view of a portion of a coil in an unexpanded state according to other embodiments hereof.

FIG. 7 shows a schematic close-up view of a portion of the coil of FIG. 4A in an unexpanded state according to embodiments hereof.

FIG. 8 shows a schematic close-up view of a portion of a coil in an unexpanded state according to other embodiments hereof.

FIG. 9 shows a schematic close-up view of a terminus of the wire of a coil according to embodiments hereof.

FIG. 10 shows a schematic close-up view of a portion of the coil of FIG. 4A on a mandrel including a terminus of the wire of the coil according to embodiments hereof.

FIGS. 11A and 11B show terminus ends of the wire of the coil of FIG. 4A according to embodiments hereof.

FIG. 12 shows a schematic side view of an expandable introducer in an expanded, unfolded state according to embodiments hereof.

### DETAILED DESCRIPTION

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components associated with, for example, a delivery device. The following detailed description is merely exemplary in nature and is not intended to limit the invention of the application and uses of the invention. Furthermore, there is no intention to be bound by any expressed or implied theory presented in the preceding field of the invention, background, summary or the following detailed description.

As used in this specification, the singular forms “a”, “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. It should be understood that use of the term “about” also includes the specifically recited number of value.

The terms “proximal” and “distal” herein are used with reference to the clinician using the devices. Therefore, “proximal” and “proximally” mean in the direction toward the clinician, and “distal” and “distally” mean in the direction away from the clinician. In other words, “proximal” and “proximally” mean towards the hub end of the introducer and “distal” and “distally” means toward the tip end of the introducer.

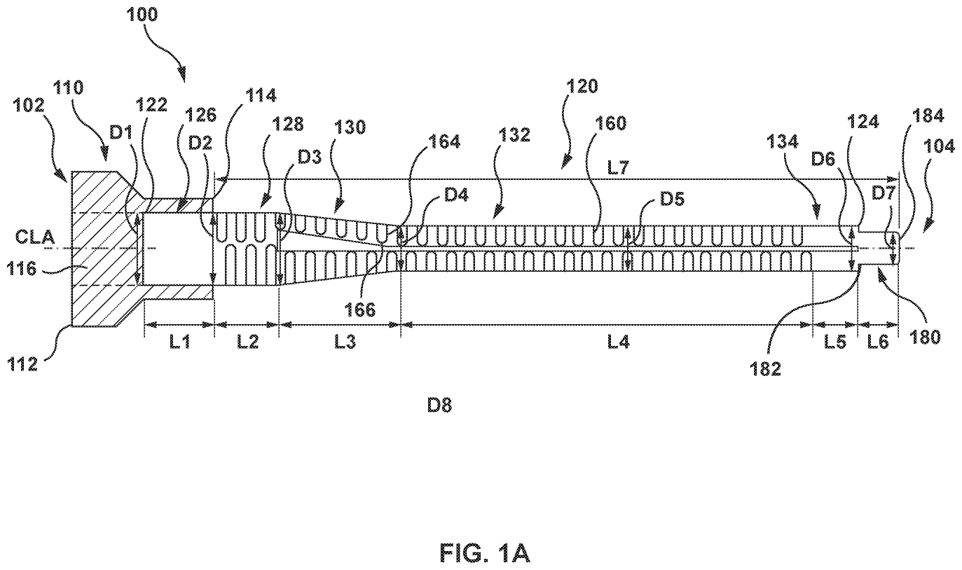

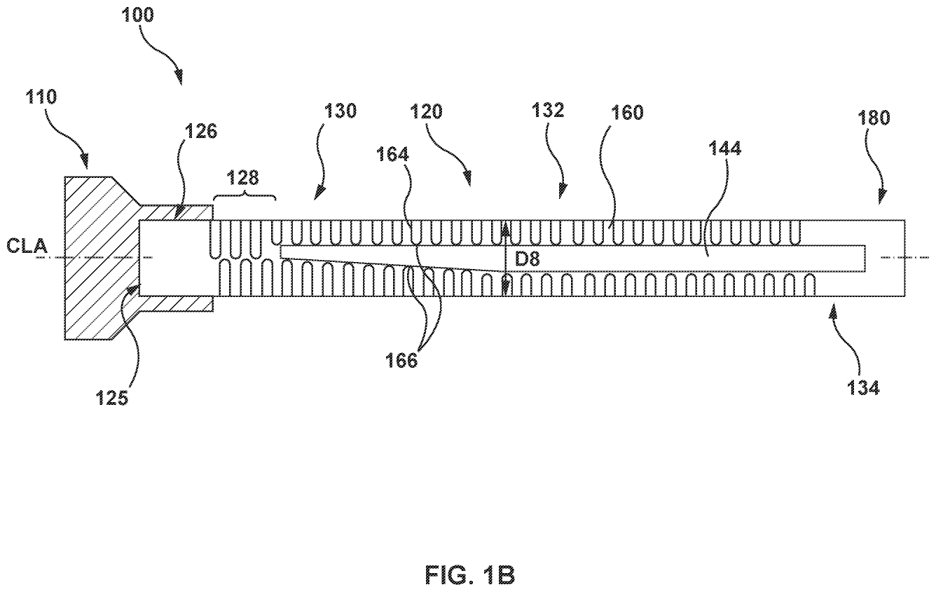

FIGS. 1A and 1B show an introducer 100 according to embodiments herein. The introducer 100 includes a proximal end 102 and a distal end 104. The introducer 100 further includes an introducer hub 110, a shaft or sheath 120, and a tip 180. The introducer hub 110 includes a proximal end 112, a distal end 114, and a hub lumen 116 extending from the proximal end 112 to the distal end 114. The sheath 120 of the introducer 100 includes a proximal end 122, a distal end 124, and a sheath lumen 125 extending from the proximal end 122 to the distal end 124. The proximal end 122 of the sheath 120 is coupled to the distal end 114 of the introducer hub 110 and extends distally therefrom. At least a portion of the sheath lumen 125 is expandable to accommodate a device (not shown) therethrough, as described in more detail below. FIG. 1A shows the sheath 120 in a radially unexpanded, folded state and FIG. 1B shows the sheath 120 in a radially expanded, unfolded state, as explained in more detail below. The sheath 120 further includes a coil 160 disposed within or embedded within the wall of the sheath 120, as described below. The coil 160 includes a series of wrap or circumferential portions 164 and a series of crowns or bends 166, as described below. One skilled in the art will realize that the introducer 100 illustrates an example of an introducer and that components may be removed, or additional components may be added. For example, and not by way of limitation, a hemostatic jacket may be provided at a proximal portion of the sheath 120.

As shown in FIG. 1A (and with reference to FIG. 1B), the tip 180 encloses, or covers, the distal end 124 of the sheath 120. With reference to the radially unexpanded, folded state of FIG. 1A and extending from the proximal end 122 to the distal end 124, the sheath 120 of the introducer 100 includes a proximal portion 126, an expanded portion 128, a tapered portion 130, an expandable coil portion 132, and an expandable non-coiled portion 134. The proximal portion 126 is disposed in a distal portion of the hub lumen 116 and is coupled thereto. The proximal portion 126 has a length L1 and a substantially uniform diameter D1. The expanded portion 128 is distal of the proximal portion 126 and is not foldable or expandable as described below for certain other portions of the sheath 120. In other words, the expanded portion 128 has a stable diameter D2 in that the diameter D2 of the expanded portion 128 does not vary between the radially unexpanded, folded state of FIG. 1A and the radially expanded, unfolded state of FIG. 1B. In the embodiment shown, the diameter D2 of the expanded portion 128 is also uniform along its length L2. In the embodiment shown, the expanded portion 128 includes a portion of the coil 160 disposed within a wall of the sheath 120 at the expanded portion, as explained below.

The tapered portion 130 extends distally from a distal end of the expanded portion 128. The tapered portion 130 has a length L3. A proximal end of the tapered portion 130 has a diameter D3 that is substantially equal to the diameter D2 of the expanded portion 128. The tapered portion 130 tapers from the diameter D3 at the proximal end of the tapered portion 130 to a diameter D4 at a distal end of the tapered portion 130, with the diameter D4 being smaller than the diameter D3. The tapered portion 130 is foldable and expandable, as explained in more detail below with respect to the expandable coiled portion 132. The tapered portion 132 includes a portion of the coil 160 in the wall of the sheath 120 at the tapered portion 132.

The expandable coiled portion 132 extends distally from the distal end of the tapered portion 130. The expandable coiled portion 132 includes a portion of the coil 160 disposed within the wall of the sheath 120, as explained below. The expandable coiled portion 132 is radially expandable from the radially unexpanded, folded state shown in FIG. 1A to the radially expanded, unfolded state shown in FIG. 1B. The expandable coiled portion 132 has a diameter D5 in the radially unexpanded, folded state that may be substantially equal to the diameter D4 at the distal end of the tapered portion 130. The diameter D5 of the expandable coiled portion 132 in the radially unexpanded, folded state is substantially uniform along the length L4 of the expandable coiled portion 132.

The expandable non-coiled portion 134 extends from a distal end of the expandable coiled portion 132. The expandable non-coiled portion 134 is radially expandable from the radially unexpanded, folded state shown in FIG. 1A to the radially expanded, unfolded state shown in FIG. 1B. The expandable non-coiled portion 134 has a diameter D6 in the radially unexpanded, folded state that may be substantially equal to the diameter D5 of the expandable coiled portion 132. The diameter D6 of the expandable non-coiled portion 134 in the radially unexpanded, folded state is substantially uniform along the length L5 of the expandable non-coiled portion 134. As implied by the naming convention, in embodiments, the expandable non-coiled portion 134 does not include a portion of the coil within a wall thereof.

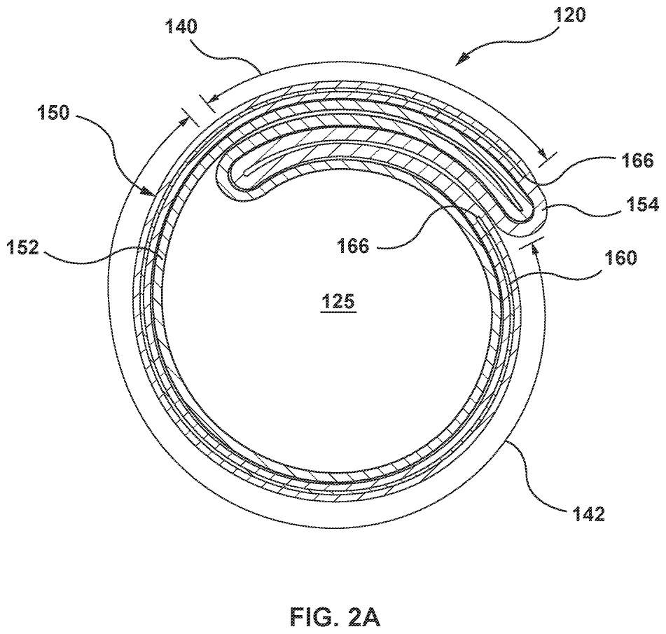

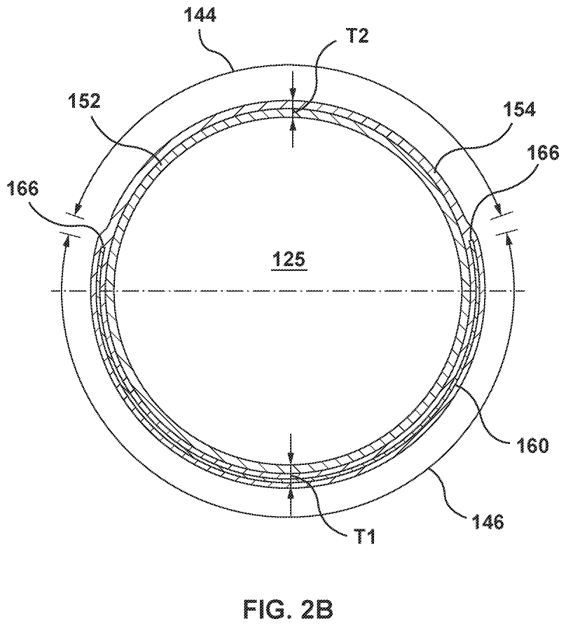

The tip 180 of the expandable introducer 100, shown in FIGS. 1A and 1B, is a tubular structure that includes a proximal end 182 and a distal end 184. The proximal end 182 is coupled to an outer surface of the distal end 124 of the sheath 120 and extends distally therefrom. The proximal end 182 of the tip 180 is coupled to an outer surface of the sheath 120 such that the tip 180 covers, or overlaps with, the distal end 124 of the sheath 120 to enable a smooth insertion transition at the distal end of the sheath 120. In particular, without the tip 180, the abrupt surface of the sheath 120 with the folded region 140 in the radially unexpanded, folded state, as shown in FIG. 2A, would be inserted into the vessel of a patient. Such an abrupt surface may cause damage to the vessel. The proximal end 182 of the tip 180 is bonded to, or coupled to, the outer surface of the sheath 120 by melt reflow of the proximal end 182 of the tip 180 with the distal end 124 of the sheath 120. The proximal end 182 of the tip 180 may also be coupled to the distal end 124 of the sheath 120 by other fusing or reflow processes. The tip 180 gradually tapers in a distal direction such that a diameter of the proximal end 182 of the tip 180 is larger than a diameter of the distal end 184 of the tip 180. The tip 180 may comprise polyether block amide (PEBAX), Polyurethane or other suitable thermoplastic polymer, and/or any combination thereof. Further details of the tip 180 may be found in International Patent Application No. PCT/US2023/061377, filed Jan. 26, 2023, which is incorporated by reference herein in its entirety.

In embodiments, the length L1 of the proximal portion 126 is approximately 19 mm, the length L2 of the expanded portion 128 is approximately 15 mm, the length L3 of the tapered portion 130 is approximately 50 mm, the length L4 of the expandable coiled portion 132 is approximately 290 mm, the length L5 of the expandable non-coiled portion 134 is approximately 10 mm, and the length L6 of the tip 180 is approximately 10 mm. However, those skilled in the art would appreciate that this is not meant to be limiting, and other lengths for the portions listed may be utilized.

In radially unexpanded, folded state, the diameters D1, D2, and D3 of the proximal portion 124, the expanded portion 126, and the proximal end of the tapered portion each may be approximately 8.7 mm, and the diameters D4, D5, and D6 at the distal end of the tapered portion 130, the expandable coiled portion 132, and the expandable non-coiled portion 134 each may be approximately 4.7 mm. The diameter D7 of the tip 180 in the radially unexpanded state may be approximately 5 to 6 mm. In the radially expanded, unfolded state, the diameter D8 of the entire sheath 120 may be approximately 8.7 mm. However, those skilled in the art would appreciate that this is not meant to be limiting, and other diameters for the portions listed may be utilized. Further, as described below, the entire sheath 120 is not necessarily in the radially expanded, unfolded state at the same time. The sheath 120 radially expands and unfolds due to a device, such as a delivery device for a prosthetic valve, being inserted through the lumen 125 of the sheath 120. Thus, at times, portions of the sheath 120 may be in the radially expanded, unfolded state, and other portions of the sheath 120 may be in the radially unexpanded, folded state, or in a state therebetween.

FIGS. 2A and 2B show an embodiment of the expandable coiled portion 132 in the radially unexpanded, folded state (FIG. 2A) and the radially expanded, unfolded state (FIG. 2B) take along lines A-A and B-B of FIGS. 2A and 2B, respectively. As shown in FIG. 2A, in the radially unexpanded, folded state, the expandable coiled portion 132 of the sheath 120 includes a folded region 140 and a non-folded region 142. As shown in FIG. 2B, the expandable coiled portion 132 of sheath 120 includes a coiled region 146 and a non-coiled region 144 around the circumference of the sheath 120.

In the embodiment of FIGS. 2A and 2B, a sheath wall 150 in the non-coiled region includes a liner 152 and an outer wall 154. In the coiled region, the sheath wall 150 includes the liner 152, the outer wall 154, and the coil embedded in the outer wall 154. In embodiments, the thickness of the outer wall 154 may vary around the circumference of the sheath wall 150. In particular, the outer wall 154 may be thinner in the non-coiled region 144 and thicker in the coiled region 146.

The liner 152 is the innermost layer of the sheath wall 150. Accordingly, an inner surface of the liner 152 defines the lumen 125 of the sheath 120. In embodiments, the liner 152 is a thin, difficult to pierce, low-friction plastic tube disposed within and coupled to the interior surface of the outer wall 154 of the sheath 120. The liner 152 is configured to reduce friction forces and aid in the translation of the device through the lumen 125 of the sheath 120 as the device is advanced distally towards the distal end 104 of the expandable introducer 100. In embodiments, the thickness of the liner 154 is about 0.05-0.15 mm and is constant, or continuous, throughout the length of the sheath 120. The liner 154 may be polytetrafluoroethylene (PTFE), fluorinated ethylene propylene (FEP) or other materials known to those skilled in the art suitable for the purposes described herein, or any combination thereof.

In the embodiment of FIGS. 2A and 2B, the outer wall 154 is a single material. In the embodiment, the outer wall 154 comprises polyether block amine (PEBAX) with a Shore D durometer value of about 63D-74D. In embodiments, the thickness of the outer wall 154 may vary such that the outer wall 154 is thinner in the

In the embodiment of FIGS. 2A and 2B, the coil 160 is disposed in the outer wall 154 in the coiled region 146 of the sheath wall 150. Details of the coil 160 are described below. The coil 160 may be embedded in the outer wall 154 of the sheath wall 150 by fusing via heat shrink, as explained below.

As shown in FIGS. 2A and 2B, the wall 150 of the sheath 120 has a varying thickness around a circumference of the sheath 120. In particular, coiled portion 146 of the wall 150 may be referred to as a thick wall region and the non-coiled region 144 may be referred to as a thin wall region of the wall 150. However, this is not meant to be limiting, and the thick wall region and the thin wall region do not need to align with the coiled portion 146 and the non-coiled portion 144. Further, the thickness may be varied gradually such that there is not an abrupt change of wall thickness. The thick wall region has a first wall thickness T1 larger than a second wall thickness T2 of the thin wall region. The reduced wall thickness in the thin wall region/non-coiled portion 144 assists in enabling the sheath 120 to fold into the radially unexpanded, folded state shown in FIG. 2A. The second thickness T2 in the thin wall region may be about 0.05-0.15 mm and the thickness T1 in the thick wall region 162 may be about 0.4-0.7 mm.

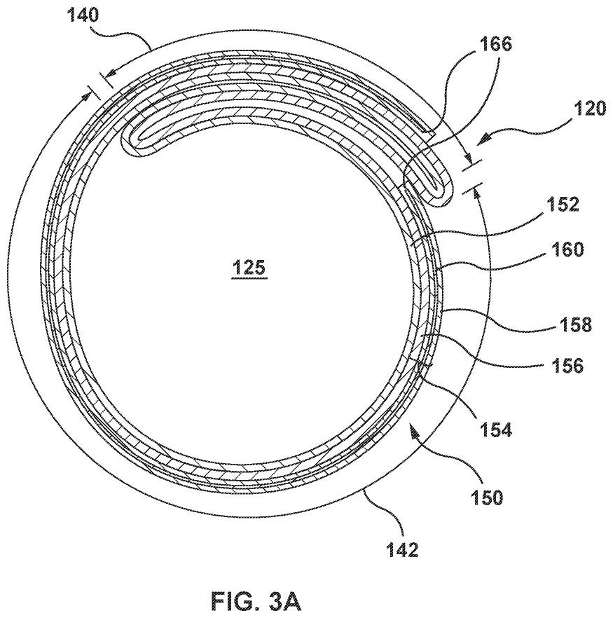

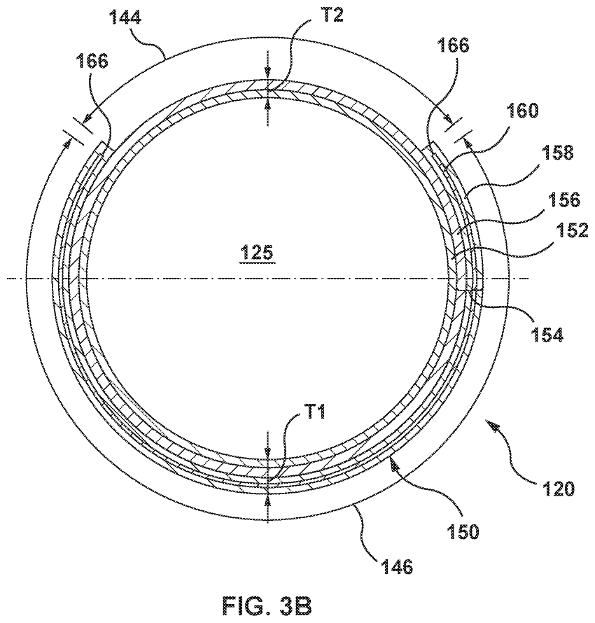

Referring to FIGS. 3A-3B, in another embodiment, the sheath wall 150 of the sheath 120 includes the liner 152, the outer wall 154, and the coil 160 embedded into the outer wall 154. However, in the embodiment of FIGS. 3A-3B, the outer wall 154 includes a first layer 158 and a second layer 156. The liner 152 may be as described above. In the embodiment of FIGS. 3A-3B, the first layer 158 of the outer wall 154 is the radially outer-most layer of the outer wall 154 and the second layer 156 of the outer wall 154 is disposed radially inward in relation to the first layer 158 of the outer wall 154, as best shown in FIG. 3B. Thus, the liner 152 of the sheath 120 is disposed inside of and coupled to an interior surface of the second layer 156 of the outer wall 154 such that the liner 152 is the radially inner-most layer of the sheath 120 and defines the lumen 125 of the sheath 121, as best shown in FIG. 3B. The first layer 158, second layer 156, and liner 152 may be formed by co-extrusion or a layered reflow lamination processes. In the embodiment shown, the first layer 158 of the outer wall 154 comprises polyether block amine (PEBAX) with a Shore D durometer value of about 63-74D. The second layer 156 of the outer wall 154 comprises PEBAX with a Shore D durometer value of about 40-63D. In an embodiment, the second layer 156 comprises PEBAX with a Shore D durometer value of about 40D. Those skilled in the art will recognize that the features of the different embodiments described with respect.

As with the embodiment of FIG. 2A-2B, the sheath 120 of the embodiment of FIGS. 3A-3B in the radially unexpanded, folded state includes a folded region 140 and a non-folded region 142, as shown in FIG. 3A. Also, in the radially expanded, unfolded state, the sheath 120 includes a coiled region 146 and a non-coiled region 144, as shown in FIG. 3B. As shown in FIG. 3B the non-coiled region 144 includes the liner 152 and the second layer 156 of the outer wall 158. The coiled region 146 includes the liner 152, the first layer 158, the second layer 156, and the coil 160 embedded within the outer wall 154. Thus, the outer wall 154, and hence the sheath wall 150, of the sheath 120 has variable wall thickness. As explained above, this variable wall thickness may be achieved with a single layer outer wall 154 instead of the two-layer outer wall 154 described with respect to FIGS. 3A-3B.

Similar to the embodiment of FIGS. 2A-2B, the wall 150 of the sheath 120 of FIGS. 3A-3B has a varying thickness around a circumference of the sheath 120. In particular, the coiled portion 146 of the wall 150 may also be referred to as a thick wall region and the non-coiled portion 144 may also be referred to as a thin wall region of the wall 150. However, this is not meant to be limiting, and the thick wall region and the thin wall region do not need to align with the coiled portion 146 and the non-coiled portion 144. Further, the thickness may be varied gradually such that there is not an abrupt change of wall thickness. The thick wall region has a first wall thickness T1 larger than a second wall thickness T2 of the thin wall region. The reduced wall thickness in the thin wall region/non-coiled portion 144 assists in enabling the sheath 120 to fold into the radially unexpanded, folded state shown in FIG. 3A. In the embodiment of FIGS. 3A-3B, the thin wall region includes the liner 152 and the second layer 156 of the outer wall 150. The thick wall region includes the liner 152, the second layer 156 and the first layer 158 of the outer wall 154, and the coil 160 embedded in the outer wall 154. Thus, the thin wall region is thinner by virtue of the first layer 158 of the outer wall 154 not being included in the thin wall region. Further, the thickness of the first layer 158 of the outer wall 154 may taper to a thinner thickness as the wall 150 transitions from the thick wall region to the thin wall region. The second thickness T2 in the thin wall region may be about 0.05-0.15 mm and the thickness T1 in the thick wall region 162 may be about 0.4-0.7 mm.

As can be seen in FIGS. 1A, 2A, and 3A, in the radially unexpanded, folded state, the coil 160 extends into the folded region 140 such that the bends/crowns 166 of the coil 160 (described below) are disposed adjacent to each other. As can been seen in FIGS. 1B, 2B, and 3B, in the radially expanded unfolded state, the bends/crown 166 of the coil 160 are spaced from each other, thereby forming the uncoiled region 144, or a gap, between the ends of the coil 160. In an embodiment, the coil 160 extends greater than 50% around the circumference of the sheath wall 150 with the sheath 120 in the radially expanded, unfolded state, as shown in FIG. 2B and 3B. For example, and not by way of limitation, in an embodiment with a diameter of the sheath 120 in the radially expanded, unfolded state of approximately 9.6 mm (circumference of approximately 30.16 mm) the arc length of the coil 160 may be approximately 17.2 mm (i.e., approximately 57% of the circumference). It has been found that the arc length of the coil 160 should be at least 15.67 mm (i.e., approximately 52% of the circumference).

As explained above, the sheath 120 is configured to radially expand from the radially unexpanded, folded state to the radially expanded, unfolded state, and radially compress back to the radially unexpanded, folded state. The sheath 120 maintains the radially unexpanded, folded state until a device is advanced through the sheath 120, during which the sheath 120 is configured to expand to the radially expanded, unfolded state. When the sheath 120 is in the unexpanded, folded state, the sheath 120 includes the folded region 140 and the non-folded region 142, as shown in FIGS. 2A and 3A. The folded region 124 is defined as the region where a section of the sheath 120 is folded over, as can be seen in FIGS. 2A and 3A. In other words, a portion of the sheath 120 is folded and bent to overlap with itself. The non-folded region 142 of the sheath 120 is defined as the remainder of the sheath 120 that does not include a fold. As can be seen in FIGS. 2A and 3A, when folded, the sheath 120 includes two folds or bends.

As explained above, the sheath 120 in the expandable coiled region 132 is configured to expand radially from the radially unexpanded, folded state having the diameter D5 to the radially expanded, unfolded state having the diameter D8 (see FIGS. 1A and 1B) larger than the diameter D5 in response to a device being advanced therethrough. As the device is advanced through the sheath 120, the folded region 140 is configured to unfold such that the sheath 120 may radially expand to the radially expanded, unfolded state, shown in FIGS. 2B and 3B. When the sheath 120 is in the radially expanded, unfolded state, there is no longer a folded region 140. In other words, the sheath 120 in the radially expanded, unfolded state has a substantially circular-shaped cross-section, as can be seen in FIGS. 2B and 3B.

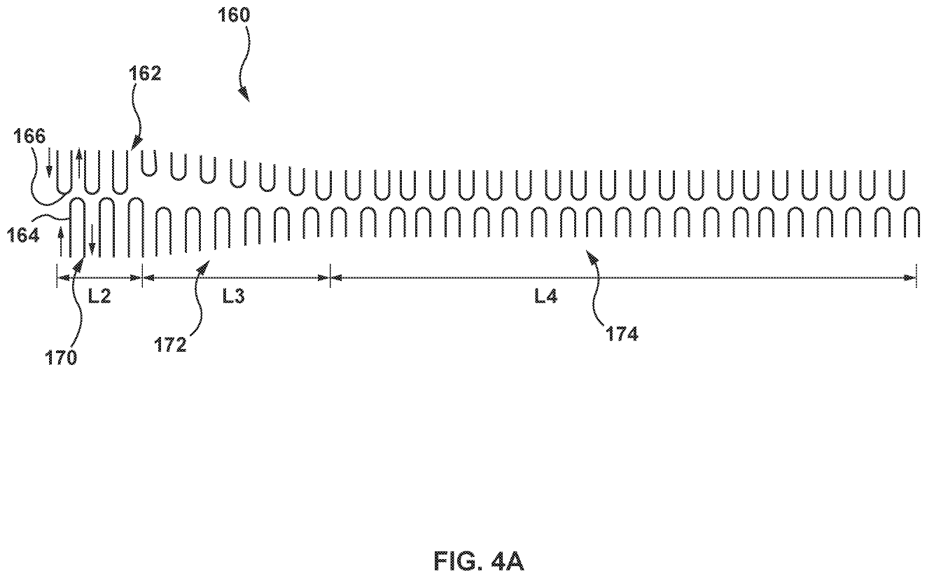

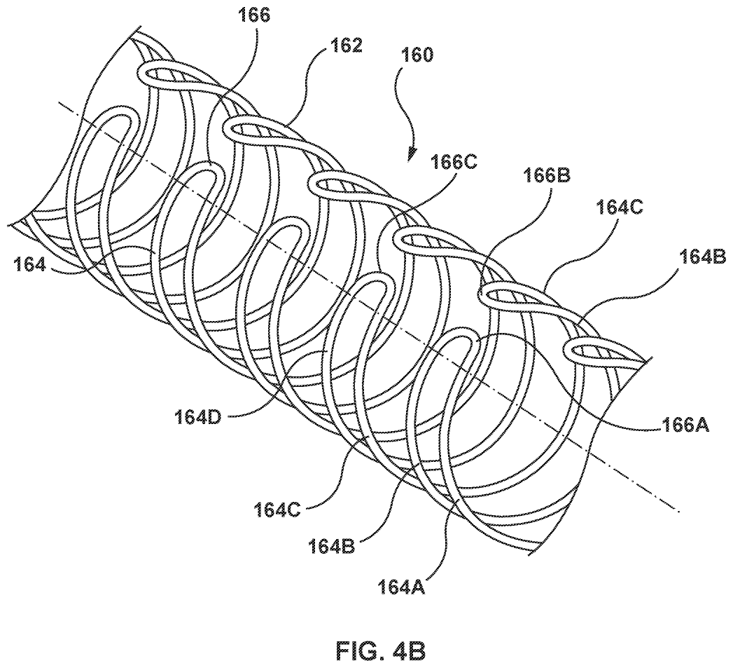

Further details of the coil 160 are described with respect to FIGS. 4A-12. FIG. 4A shows the overall pattern of the coil 160. In particular, the coil 160 in the embodiment shown includes a single wire 162 wrapped in a pattern including a series of wrap or circumferential portions 164 and a series of crowns or bends 166. The wire 162 may be 0.006 inch to 0.008 inch diameter round wire. Smaller diameter wires (e.g., 0.006 inch) embed easier in the outer wall 154 and minimize risk of protrusion of the wire 162 through the outer wall 154, whereas larger diameter wires (e.g., 0.008 inch) provide improved kink resistance for the sheath 120. The wire 162 may be a shape memory wire such that when shaped into the coil 160 and shape set, the coil 160 will return to the shape set configuration (e.g., size, shape). In an embodiment the wire 162 is a nickel-titanium alloy (e.g. Nitinol), but other superelastic or shape memory materials may be utilized.

In the embodiment shown, the coil 160 includes a proximal expanded region 170, a tapered region 172, and a distal expandable region 170, matching expanded portion 128, the tapered portion 130, and the expandable coil portion 132 of the sheath 120, described above with respect to FIGS. 1A and 1B. Accordingly, the proximal expanded portion 170 of the coil 160 is shape set to the diameter of the sheath 120 in the expanded state (e.g., approximately 8.7 mm). The coil 160 in the tapered region 172 is shape set to the tapered profile shown in FIG. 4 such that the coil 160 urges the tapered portion 130 of the sheath 120 to the tapered profile (i.e., the radially unexpanded, folded state) shown in FIG. 1A. Similarly, the distal expandable region 170 of the coil 160 is shape set to the unexpanded profile such that the coil 160 urges the expandable coil portion 132 of the sheath 120 to the radially unexpanded, folded state of FIG. 1A.

Referring to FIGS. 4A and 4B, and as noted above, the coil 160 includes circumferential portions 164 that are curved about the central longitudinal axis CLA into a C-shaped wire structure forming a series of non-continuous circumferential loops. To form the non-continuous circumferential loops, adjacent circumferential portions 164 are joined by respective bends 166, with adjacent bends 166 facing opposite directions. Thus, for example, referring to FIG. 4B, a first circumferential portion 164A wraps around the central longitudinal axis CLA in a first circumferential direction. A first bend 166A bends the wire 162 back 180 degrees such that a second circumferential portion 164B wraps around the central longitudinal axis CLA in a second circumferential direction opposite the first circumferential direction. The second circumferential portion 164B wraps around the central longitudinal axis CLA until it has wrapped almost 360 degrees around the circumference, at which point a second bend 166B bends the wire 162 back 180 degrees such that a third circumferential portion 166C wraps around the central longitudinal axis CLA in the first circumferential direction. The third circumferential portion 164C wraps around the central longitudinal axis CLA in the first circumferential direction until it has wrapped almost 360 degrees around the circumference, at which point a second bend 166C bends the wire 162 back 180 degrees such that a fourth circumferential portion 164D wraps around the central longitudinal axis CLA in the second circumferential direction. This pattern repeats itself along the length of the coil 160. As can be seen, adjacent bends 166 (such as bends 166A and 166B) are opened or face in opposite directions. The pattern of the coil 160 has been described from distal to proximal with respect to FIG. 4B, but the pattern can be described distal to proximal as well. The coil 160 may be formed by wrapping the wire 162 around a mandrel with pins located at the bends 166 in the pattern. Further, the diameter of the mandrel may match the diameter of the inner surface of the coil 160 such that that mandrel includes an expanded region, a tapered region, and an unexpanded region matching the proximal expanded region 170, the tapered region 172, and the distal expandable region 170 of the coil 160.

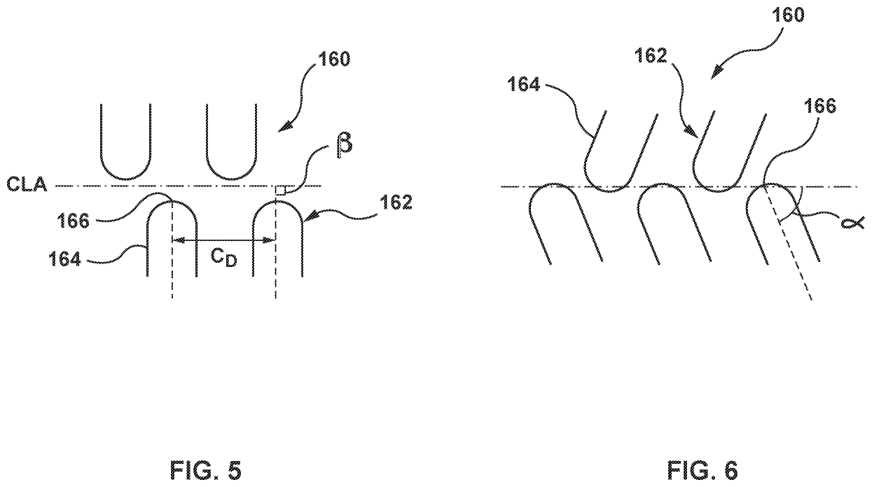

As shown in FIG. 5, the pattern of the coil 160 includes a coil pitch, that is the crown distance C_{D} between adjacent bends 166 facing the same direction. In embodiments, the crown distance C_{D} may be in the range of approximately 2.5 mm to approximately 4.5 mm. Further, as shown in FIGS. 5 and 6, the pattern of the coil 160 includes a coil angle β in the embodiment of FIG. 5 and a coil angle a in the embodiment of FIG. 6. The coil angle β in FIG. 5 is approximately 90 degrees (perpendicular) to the central longitudinal axis CLA. In other words, the circumferential portions 164 of the wire 162 extend substantially along the circumferential direction. The coil angle a in FIG. 6 may be approximately 60 degrees to approximately 120 degrees relative to the central longitudinal axis. In other words, a line that bisects the bends 166 is angled with respect to the circumferential direction. In a preferred embodiment the coil angle is substantially perpendicular to the central longitudinal axis CLA, as shown in FIG. 5.

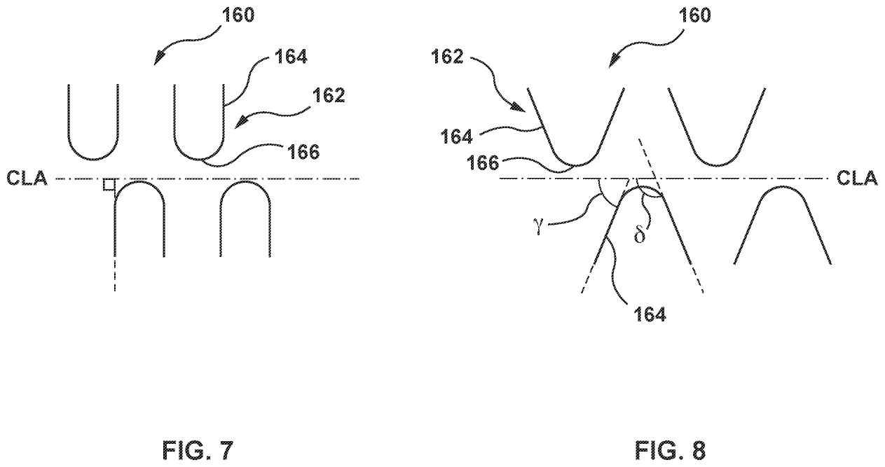

FIGS. 7 and 8 show examples of coil taper. In particular, in the embodiment of FIG. 7, as in the embodiment of FIG. 5, the circumferential portions 164 of the wire 162 are perpendicular to the central longitudinal axis CLA. In the embodiment of FIG. 8, the circumferential portions 164 are angled at a non-perpendicular angle with respect to the central longitudinal axis CLA. However, contrary to the embodiment of FIG. 6, adjacent circumferential portions 164 coupled by a bend 166 are angled such that they are splayed apart. In other words, the angles of adjacent circumferential portions 164 coupled by a bend 166 are supplementary angles y and 8 (i.e., add up to 180 degrees). The embodiment of FIG. 8 provides a coil 160 that is more stretched such that there is smaller quantity of wire in a given length of the coil 160 as compared to the embodiment of FIG. 7. The embodiment of FIG. 7 provides a more robust coil 160 for kink resistance due to the increased wire quantity and smaller gaps of unsupported sheath between the wire 162.

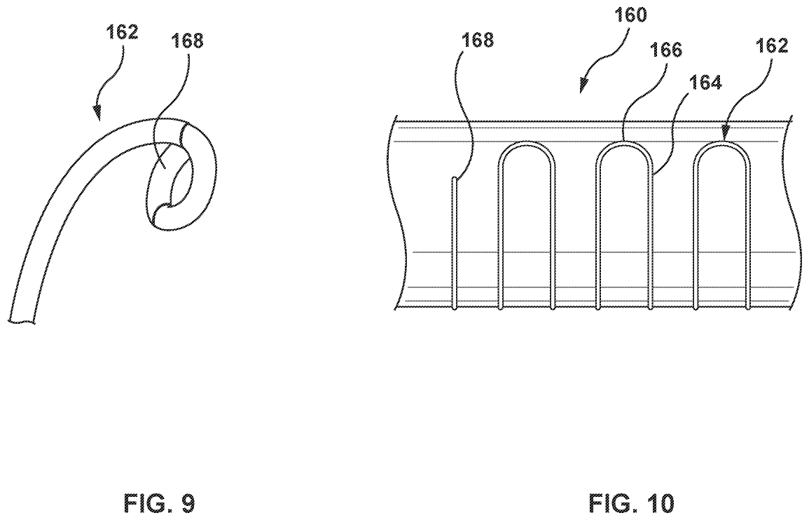

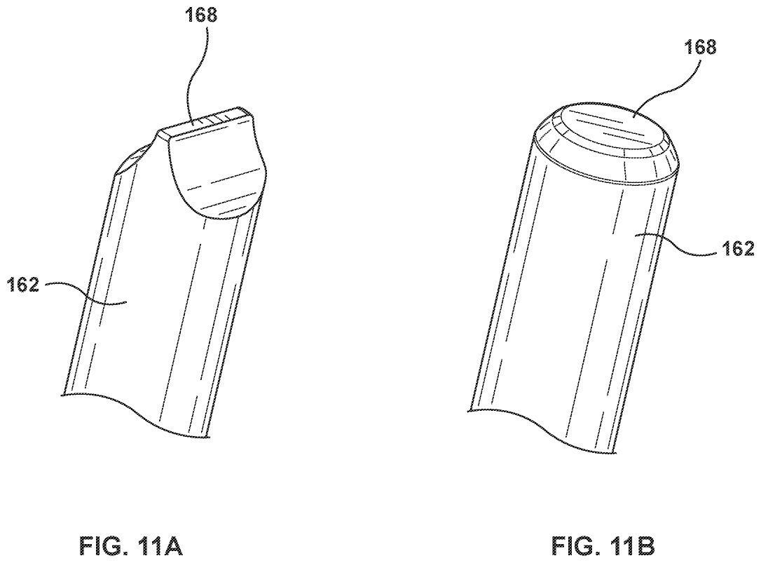

A potential issue with the coil 162, briefly mentioned above, is the wire 162 of the coil 160 protruding through the wall 150 of the sheath 120. Such protrusion through an outer surface of the wall 150 may cause damage to the surrounding vessel when the introducer 100 is inserted therein. Potential locations for such protrusions include the wire ends or termini 168. The final coil cell on each end of the coil 160 may deform during manufacture due to the free end 168 of the wire 162. FIG. 9 shows an end 168 of the wire 162. In an embodiment, the end 168 of the wire 162 is coiled around and under the wire proximal of the end 168 to create a “pigtail” loop. Such a pigtail loop places the end 168 of the wire 162 closer to the central longitudinal axis CLA and places a portion of the wire 162 outside of the end 168, thereby reducing risk of protrusion. In another embodiment, shown in FIG. 10, the end 168 of the wire is biased towards the inner surface of the wall 150, thereby reducing risk of protrusion. In another embodiment, shown in FIG. 11B, the end 168 of the wire 162 is laser cut, leaving a smooth, rounded profile of the end 168. As can be seen in comparison to FIG. 11A, which shows the end 168 cut using a snip tool, the end 168 in FIG. 11B is smoother than the end 168 of FIG. 11A. Such a smooth end mitigates the risk of a sharp edge penetrating the wall 150.

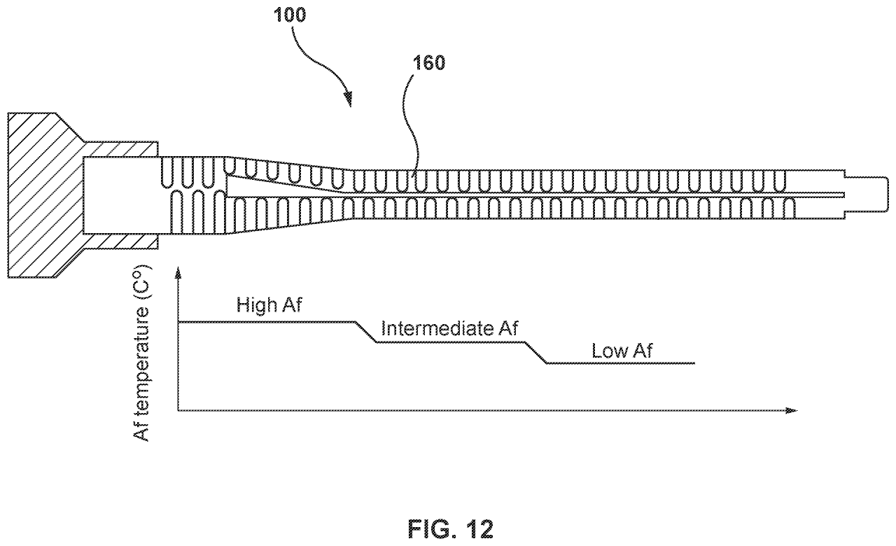

FIG. 12 shows an embodiment of the introducer 100 according to another embodiment hereof. The introducer 100 of FIG. 12 is substantially similar to the introducer 100 of FIGS. 1A and 1B. Therefore, the details of the introducer 100 of FIG. 12 will not be repeated and the details described with respect to FIGS. 1A and 1B, and FIGS. 2A through 11 are incorporated into the description of FIG. 12, unless specifically excluded. As explained above, the wire 162 is a shape memory material such that the coil 160 may be shape set to return to the radially unexpanded state such that the sheath 120 may by shape set to return to the radially unexpanded, folded state after the device has been removed. Shape memory materials, such as Nitinol, have an Active A(f) temperature AF above which the shape memory material returns to the shape that was shape set. Therefore, for example, a shape memory material with an Active A(f) temperature AF of 20 degrees Celsius will exhibit shape recovery above 20 degrees Celsius. In the embodiment of FIG. 12, the different sections of the coil 160 may have different AF temperatures to vary the performance of the coil 160 along the length of the sheath 120. For example, in the example of FIG. 12, the proximal expanded region 170 and tapered region 172 of the coil has a first AF temperature, a proximal portion of the expandable region 174 has a second AF temperature, and a distal portion of the expandable region 174 has a third AF temperature. In the embodiment shown, the first AF temperature is higher than the second AF temperature, which is higher than the third AF temperature. Thus, the AF temperature is reduced from a proximal end of the coil 160 to a distal end of the coil 160. Those skilled I the art will recognize that the transition locations for the AF temperatures described with respect to FIG. 12 are not meant to be limiting, and there may be more or fewer than three AF temperatures. For example, and not by way of limitation, the second and third AF temperatures in FIG. 12 may be the same such that there are only two AF temperatures. In another example, the first AF temperature may comprise two AF temperatures with a transition at the tapered region 172 such that there are four AF temperatures. As described, in a preferred embodiment, the AF temperature decreases distally. In such an embodiment, the force required to expand the coil 160, and hence the sheath 120, is lower in the higher AF temperature regions and higher it the lower AF temperature regions. Thus, in the embodiment of FIG. 12, the force required to expand the coil 160, and hence the sheath 120, is lower in the proximal portions of the sheath 120 and higher in the distal portions of the sheath 120. Such an arrangement may be beneficial due to insertion forces at the proximal portion of the sheath 120 generally being higher than in the distal portion of the sheath 120. Thus, the coil 160 can be optimized to reduce the insertion forces where the insertion forces tend to be the highest.

As briefly described above, the sheath 120 with the coil 160 may be formed via heat shrink fusing. In one example, the liner 152 may be mounted over a fuse mandrel and the second layer 156 of the outer wall 154 is loaded over the liner 152. The coil 160 is then loaded over the second layer 156. The first layer 158 is then placed over the coil 160 and the second layer 156, recognizing that the first layer 158 is not a complete tube, as shown in FIG. 3B. A heat shrink may then be loaded over the entire assembly and the layers may be fused together in a laminator.

In another embodiment, the coil 160 is loaded over the fuse mandrel. The first layer 158 is loaded over the coil 160. A heat shrink is loaded over the first layer 158, and the assembly is fused in a laminator to embed the coil 160 into the first layer 158. The material of the first layer 158 may be removed to form the gap in the first layer 158 shown in FIG. 3B. Next, the first layer 158 with the coil embedded therein may be removed from the fuse mandrel. The liner 152 may be loaded over the fuse mandrel, the second layer 156 may be loaded over the liner 152, and the first layer 158 with the coil 160 embedded therein may be loaded over the second layer 156. A heat shrink is loaded over the full assembly and the layers are fused together in a laminator.

The manufacturing embodiments described above are with respect to the embodiment of FIGS. 3A-3B, which includes two layers for the outer wall 154. Similar processes may be used for the embodiment of FIGS. 2A-2B. In particular, in an embodiment, the liner 152, the coil 160, and the outer wall 154 are loaded onto the fuse mandrel. A heat shrink is loaded over the liner 152, coil 160, and outer wall 154 assembly. The layers are fused together in a laminator. In another embodiment, the coil 160 and the outer wall 154 are loaded on the fuse mandrel. A heart shrink is loaded over the coil 160 and the outer wall 154, and the assembly is fused together using a laminator. The coil 160/outer wall 154 fused assembly in removed from the fuse mandrel. The liner 152 is loaded onto the fuse mandrel and the coil 160/outer wall 154 fused assembly is loaded over the liner 152. A heat shrink is loaded over the liner 152 and coil 160/outer wall 154 assembly, and the assembly is fused together using a laminator.

While some examples of embedding the coil 160 into the wall 150 of the sheath 120 are described, these are not mean to be limiting, and other ways to embed the coil 160 into the wall 150 are also contemplated, as would be understood by those skilled in the art.

The above description is directed to the sheath 120 of the introducer 100. As explained above, additional or different components may be used with the sheath 120 of the introducer 100. For example, and not by way of limitation, the hub 110 has not been described in detail, as hubs 110 known to those skilled in the art may be utilized. Similarly, additional components, such as, but not limited to, hemostatic jackets, tips, and hubs may be added and/or substituted for components of the introducer 100. In particular, the components described in International Patent Application No. PCT/US2023/061377, filed Jan. 26, 2023, are incorporated by reference herein in their entirety.

As used herein, the term “generally” and “substantially” mean approximately. When used to describe angles such as “substantially parallel” or “substantially perpendicular” the term “substantially” means within 10 degrees of the angle. When used to describe shapes such as “substantially” or “generally” cylindrical or “substantially” or “generally” tube-shaped or “generally” or “substantially” conical, the terms mean that the shape would appear cylindrical or tube-shaped or conical to a person of ordinary skill in the art viewing the shape with a naked eye. The term “about” as used herein to refer to dimensions means within 5% of the dimension.

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components.

What is claimed is:

1. An expandable introducer comprising:
a hub having a distal end and a proximal end; and
a sheath coupled to the hub and extending distally therefrom, the sheath defining a central lumen, the sheath configured to radially expand from a radially unexpanded, folded state having a first diameter and to a radially expanded, unfolded state having a second diameter larger than the first diameter in response to a device passing through the central lumen, wherein a wall of the sheath includes a thick wall region having a first wall thickness and a thin wall region having a second wall thickness smaller than the first wall thickness, wherein the thick wall region includes a coil embedded within the wall of the sheath.

2. The expandable introducer of claim 1, wherein the first wall thickness is about 0.4-0.7 mm.

3. The expandable introducer of claim 1, wherein the second wall thickness is about 0.05-0.2 mm.

4. The expandable introducer of claim 1, wherein:
the coil is a C-shaped coil including a plurality of circumferential portions coupled by bends.

5. The expandable introducer of claim 4, wherein adjacent bends of the coil face opposite circumferential directions such that there is a circumferential gap between the bends facing opposite directions in the radially unexpanded, folded state.

6. The expandable introducer of claim 1, wherein the coil is a shape memory material and is shape set to the radially unexpanded, folded state.

7. The expandable introducer of claim 1, wherein the sheath comprises a proximal portion disposed within a lumen of the hub, an expanded portion distal of the proximal portion, a tapered portion distal of the expanded portion, an expandable coiled portion distal of the tapered portion, and an expandable non-coiled portion distal of the expandable coiled portion.

8. The expandable introducer of claim 7, wherein the expanded portion is not foldable and is the second diameter.

9. The expandable introducer of claim 7, wherein the tapered portion tapers in the distal direction such that in the radially unexpanded, folded state of the sheath, a proximal end of the tapered portion has the second diameter and a distal end of the tapered portion has the first diameter.

10. The expandable introducer of claim 9, wherein in the radially expanded, unfolded state, both the proximal end and the distal end of the tapered portion have the second diameter.

11. The expandable introducer of claim 7, wherein the expandable coiled portion has the first diameter in the radially unexpanded, folded state and the second diameter in the radially expanded unfolded state.

12. The expandable introducer of claim 7, wherein the expandable non-coiled portion has the first diameter in the radially unexpanded, folded state and the second diameter in the radially expanded, unfolded state.

13. The expandable introducer of claim 7, wherein the coil is embedded in the wall of the sheath along the expanded portion, the tapered portion, and the expandable coiled portion.

14. The expandable introducer of claim 7, wherein the coil is not embedded in the wall of the sheath along the proximal portion and the expandable non-coiled portion.

15. The expandable introducer of claim 7, wherein the coil is a shape memory material, and wherein an Active A(f) temperature of the coil is higher in a proximal region of the coil than in a distal region of the coil.

16. The expandable introducer of claim 7, wherein the coil includes a proximal expanded region embedded in the expanded portion of the sheath, a tapered region embedded in the tapered portion of the sheath, and a distal expandable region embedded in the expandable coiled portion of the sheath.

17. The expandable introducer of claim 16, wherein the proximal expanded region and the tapered region of the coil have a first Active A(f) temperature and the distal expandable region of the coil has a second Active A(f) temperature lower than the first Active A (f) temperature.

18. The expandable introducer of claim 1, wherein the coil comprises a wire, wherein at least one end of the wire is formed into a pigtail.

19. The expandable introducer of claim 1, wherein the coil comprises a wire, wherein at least one end of the wire is disposed closer to a central longitudinal axis of the sheath than a middle portion of the wire.

20. The expandable introducer of claim 1, wherein the coil comprises a wire, wherein at least one end of the wire is laser cut such that the at least one end has a smooth profile.
